(19) United States
(12) Patent Application Publication
(10) Pub. No.: US 2007/0167969 A1
(43) Pub. Date: Jul. 19, 2007

(54) Surgical cutting tool for making precise and accurate incisions

(76) Inventor: Rajesh Pandey, Plantation, FL (US)

Correspondence Address: COOPER & DUNHAM, LLP, 1185 AVENUE OF THE AMERICAS, NEW YORK, NY, 10036, US

(21) Appl. No.: 11/332,455

(22) Filed: Jan. 13, 2006

(51) Int. Cl. A61B 17/34

(52) U.S. Cl. 606/185

(57) ABSTRACT

A tool for making a cut in a patient's body. The tool includes a housing including a first end, a second end, and having an axis. A shaft is positioned moveably along the axis. A blade is attached to the shaft. An interface is attached to one end of the housing, which is shaped and dimensioned to communicate with a connector attached to a patient's body.

### BACKGROUND

A heart pump, such as a ventricular assist device (“VAD”), aids people suffering from severe ventricular heart failure in leading active and productive lives. A heart pump is typically connected to the left ventricle of the heart. One end of a tube, such as a graft, is connected to the heart pump and the other end is connected to the ascending aorta or the descending aorta. Once connected, the heart pump pumps blood from the left ventricle to the ascending or descending aorta to improve blood flow.

To connect a heart pump to a patient, surgeons use a connector, called a sewing ring. A sewing ring attaches to the myocardium of the heart through the use of sutures. A hole is then cored in the myocardium that acts as an entry site. An inflow tube from the heart pump is inserted through this hole. For the implantation to be successful, however, the cored hole must be centered and sized appropriately so that there is minimal leaking between the ventricular wall and the inflow tube.

To initiate coring, a surgeon must first make a manual “cross” or “crux” cut in the ventricle wall. A coring tool is then inserted through the crux cut and used to form the hole. If the surgeon is not careful, the manual “crux” cut might end up longer than the diameter of the coring tool, or it might be off-center with respect to the sewing ring. If either of these situations occur, leaking could occur between the inflow tube and the ventricular wall. To prevent this from occurring, surgeons generally overcompensate by making cross cuts smaller than necessary. This has the drawback, however, of making the coring tool much more difficult to insert in the crux cut. Accordingly, what is needed is a cutting tool that allows surgeons to make accurate and precise crux cuts.

### SUMMARY OF THE INVENTION

In one embodiment, the present invention comprises a surgical tool for making a cut in a patient's body. The tool comprises a housing including a first end, a second end, and having an axis. A shaft is moveably positioned along the axis. A blade is attached to the shaft. An interface is attached to the second end of the housing. The interface is shaped and dimensioned to communicate with a connector attached to a patient's body.

In one embodiment, the housing comprises a handle portion and a blade receiving portion. The handle portion comprises a cylindrical shaft with an outer diameter.

In one embodiment, the blade receiving portion comprises a frustroconical section having a first end with an outer diameter and a second end with an outer diameter. The first frustroconical section end is attached to the cylindrical shaft and the outer diameter of the first frustroconical section end is approximately equal to the outer diameter of the cylindrical shaft. The outer diameter of the second frustroconical section end is greater than the outer diameter of the cylindrical shaft.

In one embodiment, the blade receiving portion includes a cylindrical section attached to the second frustroconical section end. The cylindrical section has an outer diameter that is equal to the outer diameter of the second frustroconical section end. The interface comprises a cylindrical lip attached to the cylindrical section. The cylindrical lip has an outer diameter that is less than the outer diameter of the cylindrical section. The blade is centered with respect to the cylindrical lip.

In one embodiment, a ridge is positioned on the handle portion that provides a surface for a user's fingers when making a cut.

In one embodiment, a resilient member is positioned between the housing and the shaft. The resilient member is operative to bias the shaft away from the interface.

In one embodiment, the shaft includes a first end and a second end. The first shaft end extends at least partially beyond the first housing end. The first shaft end acts as an actuating mechanism. The second shaft end is attached to the blade.

In one embodiment, the blade is a triangular shaped blade.

In one embodiment, the blade is a first triangular shaped blade attached to a second triangular shaped blade in a perpendicular relationship.

In a further embodiment, a method is provided. A cutting tool is provided that includes a housing with an interface shaped and dimensioned to communicate with a connector attached to a patient's body. The interface communicates with a connector positioned on the patient's body. The cutting tool is actuated to create an incision on the patient's body.

In one embodiment, the cutting tool is removed from engagement with the connector. The cutting tool is rotated 90 degrees. The interface is mated with the connector. The cutting tool is actuated to create another incision on the patient's body.

In one embodiment, the housing has an axis. The cutting tool is provided with a shaft positioned along the axis in the housing. The shaft includes a first end and a second end. A blade is attached to the second end of the shaft. Actuating the cutting tool involves gripping the housing and depressing the first end of the shaft such that the blade extends from the second end of the housing.

In one embodiment, a spring is provided between the shaft and the housing. Pressure is removed from the first end of the shaft and the spring is allowed to retract the blade within the housing.

In one embodiment, the connector is a sewing ring. The cutting tool is provided with an interface that is a circular lip attached to the housing. The registering the circular lip is registered with the sewing ring and the circular lip is inserted within a space defined by the sewing ring.

Other objects, features and advantages of the invention will be apparent from the following detailed disclosure, taken in conjunction with the accompanying sheets of drawings, wherein like numerals refer to like parts, elements, components, steps and processes.

### BRIEF DESCRIPTION OF THE DRAWINGS

FIG. 1 is a frontal view of a sewing ring attached to a portion of human heart with a crux cut shown centered therein.

FIG. 2 is a perspective view of a crux cutter with the blade shown retracted.

FIG. 3 is a perspective view of the crux cutter of FIG. 2 with the blade shown extended.

FIG. 3a is a perspective view of an alternate embodiment of the blade shown in FIG. 3.

FIG. 4 is an exploded view of the crux cutter of FIG. 2.

FIG. 5 is an elevated sectional view taken along line 5-5 of FIG. 4.

FIG. 6 is an elevated enlarged view of the blade shown in FIG. 5.

FIG. 7 is an elevated enlarged side view of the blade shown in FIG. 5.

### DETAILED DESCRIPTION

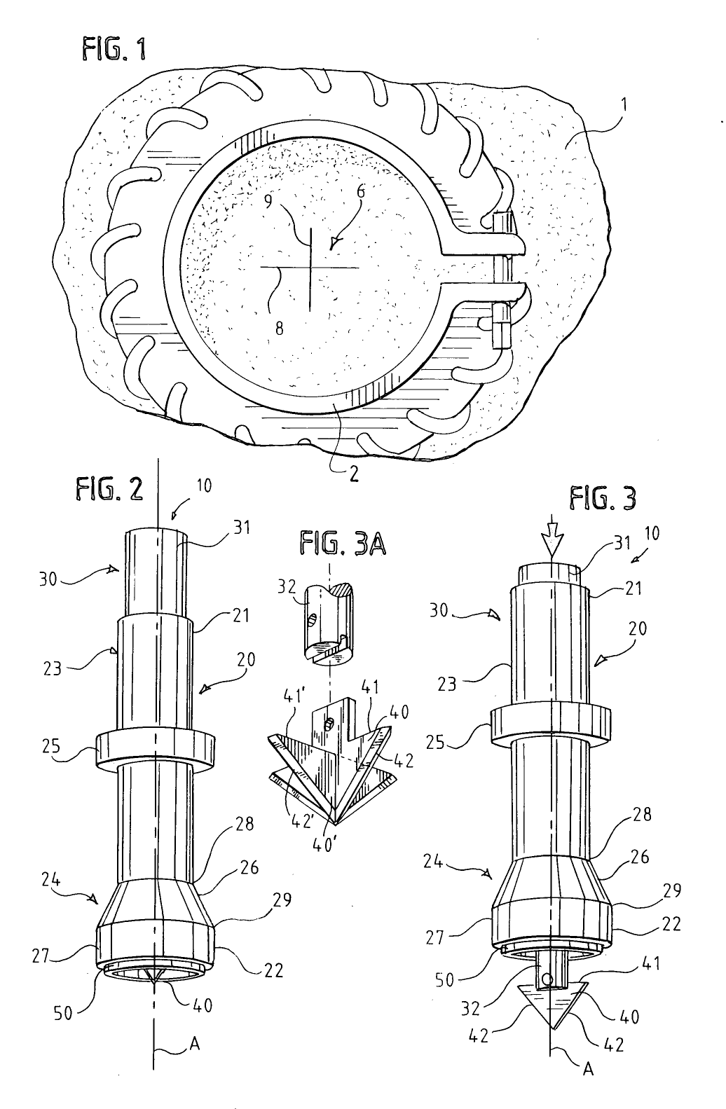

Referring to FIG. 1, a portion of a ventricle wall 1 is shown with a connector 2 for a VAD attached. In one example, the connector 2 is a sewing ring that is attached to the ventricle wall through suturing. An exemplary connector can be found in the United States patent application filed Dec. 8, 2005 for the invention entitled “Implant Connector” and naming Daniel Tamez, Vitrote lndravudh, Richard A. Marquis, Charles R. Shambaugh and Jeffrey A. LaRose as inventors, and having Seyfarth Shaw LLP Docket No. 25658400400, the disclosure of which is hereby incorporated by reference.

In order to connect a VAD to the ventricle wall 1, a suitable hole must be formed in the space 4 defined by sewing ring 2. In one example, a surgeon would form such a hole using a coring tool. An exemplary coring tool can be found in the United States patent application filed at the U.S. Patent and Trademark Office on the same date as the present application, and naming Rajesh Pandey as inventor and entitled “Surgical Tool for Coring Precise Holes and Providing for Retrieval of Tissue”, Seyfarth Shaw LLP, Docket No. 25658-400900, the disclosure of which is hereby incorporated by reference. Ultimately, the hole formed in the ventricle wall 1 must be of a suitable size and shape to receive an inflow tube from the VAD. An exemplary hole would be one of generally circular shape and having a diameter of 15.7 mm, although the size and the shape of the hole will vary depending on the size and shape of the inflow tube on the VAD.

Before using a coring tool to form the hole, a surgeon must first form a “crux cut” 6 in the ventricle wall. The coring tool is then inserted into the crux cut 6 and the hole is formed. The crux cut 6 is formed from a first incision 8 and a second incision 9. The first incision 8 and second incision 9 intersect to form a cross, hence the name “crux cut”.

Referring to FIGS. 2 and 3, an exemplary embodiment of a novel cutting tool 10 for forming a crux cut is shown for illustrative purposes. The cutting tool 10 in one example comprises a housing 20, a shaft 30, a blade 40, and an interface 50 for piloting into and engaging sewing ring 2 (FIG. 1). In one example, the components of the cutting tool 10 are made of the same material (e.g. stainless steel). In another example, housing 20, shaft 30, and interface 50 are made of one material, such as plastic, and blade 40 is made of another material, such as stainless steel. Other combinations of materials are also possible depending on the needs of the end users and/or manufacturers.

In one example, the housing has two opposing ends 21, 22 and an axis A that runs from end 21 to end 22. The shaft 30 is slidably positioned in housing 20 and moves axially with respect to housing 20. The housing 20 has a handle portion 23 and a blade receiving portion 24. The handle portion 23 in one example is a cylindrical shaft having a ridge 25 disposed thereon. A surgeon's fingers can rest on the ridge 25, and a surgeon can use the ridge 25 to gain leverage to apply downward pressure, if necessary, when making a cut. The blade receiving portion 24, in one example, comprises a frustroconical section 26 and a cylindrical section 27. The frustroconical section 26 section has a first end 28 and a second end 29. The outer diameter of the second end 29 is greater than the outer diameter of the first end 28. An exemplary value for the outer diameter of the first end is 0.620 inches. An exemplary value for the outer diameter of the second end 29 is 0.960 inches.

In one example, the outer diameter of the handle portion 23 is equal to the outer diameter of the first end 28 of frustroconical section 26, and the outer diameter of the cylindrical section 27 is equal to the outer diameter of the second end 29. The first end 28 of the frustroconical section 26 is attached to the handle portion 23. The second end 29 of frustroconical section 26 is attached to the cylindrical section 27. In the example shown, handle portion 23, ridge 25, frustroconical section 26, and cylindrical section 27 are formed integrally as one piece. In another example (not shown), these sections could be multiple pieces that connected together through suitable connecting means, such as press fits, laser welding, or slip-fits held with set screws.

Referring further to FIGS. 2-4, shaft 30 is slidably positioned within housing 20 and is axially moveable with respect to housing 20. Shaft 30 includes first end 31 and a second end 32. Shaft 30 is sufficiently long that first end 31 extends out of housing 20 when blade 40 is in a retracted position (FIG. 2) or an extended position (FIG. 3). Accordingly, first end 31 acts as a button that can actuate crux cutter 10 by pushing first end 31 toward the second end 22 of housing 20, thereby extending blade 40 from housing 20. An exemplary length for shaft 30 is 4.180 inches given an exemplary length for housing 20 of 3.075 inches.

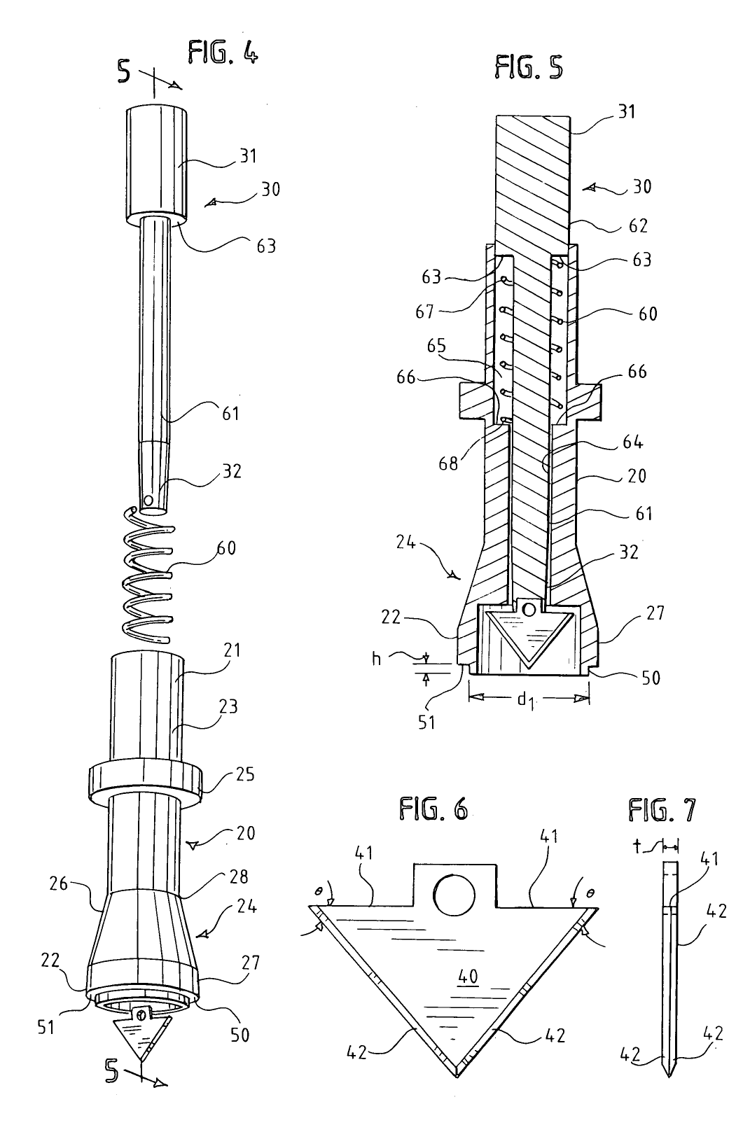

Blade 40 is attached to the second end 32 of shaft 30. In one example, the blade 40 is attached to the shaft 30 by a button head cap screw that is threaded onto shaft 30. Other suitable connecting means, such as gluing or laser welding can also be used. Referring to FIG. 6, blade 40, in one example, is triangular in shape with a base 41 having a length of 16 mm and two sides 42, each having an angle of Θ (128) degrees from base 41 and having a length of 0.510 inches. Referring to FIG. 7, in one example blade 40 has a thickness of 0.30 inches. In another example, shown in FIG. 3A, blade 40 could comprise two blades 40, 40′ arranged perpendicularly in a diamond shape. Each blade 40, 40′ would have a base 41, 41′ and two sides 42, 42′.

Referring further to FIGS. 4-5, interface 50 in one example comprises a cylindrical lip that extends out from the open end of the blade receiving portion 24 of housing 20. In one example, cylindrical lip has an outer diameter d₁ of 0.815 inches and a height h of 0.063 inches for use with a sewing ring having the dimensions of 0.830 inches (dia) and 0.070 inches. The lip attaches to a bottom edge 51 of the cylindrical section 27 of the blade receiving portion 24. Interface 50 is shown as formed integral as one piece with housing 20, but it is envisioned that interface 50 could be a separate component that is affixed to housing 20 through other means or it could be retrofitted to an existing tool.

Referring further to FIGS. 4-5, in one example, blade 40 is auto retractable through the provision of a resilient element, such as spring 60 disposed between housing 20 and shaft 30. Shaft 30 is shown as a cylinder having a first portion 61 and a second portion 62 in which the first portion 61 has a smaller diameter than the second portion 62. Exemplary diameters for the shaft 30 are 0.260 inches for the first portion 61 and 0.500 inches for the second portion 62. The difference in diameters forms a ridge 63. Similarly, housing 20 includes a first recess 64 and a second recess 65, in which the first recess 64 and the second recess 65 are cylindrically shaped and having diameters such that the diameter of the first recess 64 is less than the diameter of the second recess 65. Exemplary diameters are 0.503 inches for the first recess 64 and 0.280 inches for the second recess 65. The difference in recess diameters forms another ridge 66. Spring 60 has a first end 67 that is positioned in engagement with ridge 63 and a second end 68 that is positioned in engagement with ridge 66. Accordingly, shaft 30 and blade 40 are biased by spring 60 away from the second end 22 of housing and thus in a retracted position.

Referring to FIGS. 1 and 2, to use the cutting tool 10, a surgeon or other user will first attach a connecting device, such as a sewing ring 2, to a patient's ventricle 1. The surgeon will then pilot the cutting tool 10 into the space 4 formed by the sewing ring 2 by communicating the interface 50 with the sewing ring 2. The surgeon will, if necessary, apply downward pressure on the ridge 25 by using his fingers. The surgeon will actuate the cutting tool 10 by pressing the end 32 of shaft 30 while holding the housing 20. The blade 40 will plunge into the ventricle thereby making a first incision 8. The surgeon will release the pressure on end 32 of shaft 30, thereby causing the spring to retract blade 40. The surgeon disengages the cutting tool 10 from the sewing ring 2 and rotates the cutting tool 90 degrees. The surgeon will then repeat the preceding procedure to form a second incision 9. In this manner, crux cut 6 is formed. In the alternative embodiment, shown in the inset of FIG. 3A, a diamond shaped blade 40 is used. Therefore, the surgeon will not have to remove the cutting tool 10 and rotate it 90 degrees because a crux cut will be formed after the first actuation of cutting tool 10.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A surgical tool for making a cut in a patient's body, the tool comprising:
a housing including a first end, a second end, and having an axis;
a shaft moveably positioned along the axis;
a blade attached to the shaft; and
an interface attached to the second end of the housing, wherein the interface is shaped and dimensioned to communicate with a connector used to connect a medical device to a patient's body.

2. The surgical tool of claim 1, wherein the housing comprises a handle portion and a blade receiving portion.

3. The surgical tool of claim 2, wherein the handle portion comprises a cylindrical shaft with an outer diameter.

4. The surgical tool of claim 3, wherein the blade receiving portion comprises a frustroconical section having a first end with an outer diameter and a second end with an outer diameter, wherein the first frustroconical section end is attached to the shaft and the outer diameter of the first frustroconical section end is approximately equal to the outer diameter of the cylindrical shaft.

5. The surgical tool of claim 4, wherein the outer diameter of the second frustroconical section end is greater than the outer diameter of the cylindrical shaft.

6. The surgical tool of claim 5, wherein the blade receiving portion further comprises:
a cylindrical section attached to the second frustroconical section end, wherein the cylindrical section has an outer diameter that is equal to the outer diameter of the second frustroconical section end.

7. The surgical tool of claim 6, wherein the interface comprises a cylindrical lip attached to the cylindrical section.

8. The surgical tool of claim 7, wherein the cylindrical lip has an outer diameter that is less than the outer diameter of the cylindrical section.

9. The surgical tool of claim 7, wherein the blade is centered with respect to the cylindrical lip.

10. The surgical tool of claim 2, further comprising:
a ridge positioned on the handle portion, wherein the ridge provides a surface for a user's fingers when making a cut.

11. The surgical tool of claim 1, further comprising:
a resilient member positioned between the housing and the shaft, the resilient member operative to bias the shaft away from the interface.

12. The surgical tool of claim 1, wherein the shaft includes a first end and a second end, the first shaft end extending at least partially beyond the first housing end, such that the first shaft end acts as an actuating mechanism, and wherein the second shaft end is attached to the blade.

13. The surgical tool of claim 1, wherein the blade comprises at least one triangular shaped blade.

14. The surgical tool of claim 13, wherein the blade comprises a first triangular shaped blade attached to a second triangular shaped blade.

15. The surgical tool of claim 14, wherein the first triangular shaped blade and the second triangular shaped blade are oriented perpendicular to each other.

16. A method of making a cut on a patient's body, the method comprising:
providing a cutting tool that includes a housing, and an interface connected to the housing that is shaped and dimensioned to communicate with a connector attached to a patient's body;
communicating the interface of the cutting tool with the connector attached on the patient's body; and
actuating the cutting tool to create an incision on the patient's body.

17. The method of claim 16, further comprising:
removing the cutting tool from communication with the connector;
rotating the cutting tool 90 degrees;
communicating the interface of the cutting tool with the connector; and
actuating the cutting tool to create another incision on the patient's body.

18. The method of claim 16, wherein the housing has an axis and the step of providing the cutting tool comprises:
providing the cutting tool with a shaft positioned along the axis in the housing, wherein the shaft includes a first end and a second end;
providing a blade that is attached to the second end of the shaft.

19. The method of claim 18, wherein the step of actuating comprises:
gripping the housing; and
depressing the first end of the shaft such that the blade extends from the second end of the housing.

20. The method of claim 19, further comprising:
providing a spring between the shaft and the housing;

21. The method of claim 20, further comprising:
removing pressure from the first end of the shaft; and
allowing the spring to retract the blade within the housing.

22. The method of claim 16, wherein the connector is a sewing ring, and wherein the step of providing the cutting tool comprises:
providing the interface as a circular lip attached to the housing.

23. The method of claim 22, wherein the step of communicating the interface with the connector comprises:
registering the circular lip with sewing ring; and
inserting the circular lip within a space defined by the sewing ring.
